(19) United States
(12) Patent Application Publication
(10) Pub. No.: US 2009/0098064 A1
(43) Pub. Date: Apr. 16, 2009

(54) METHOD OF TREATING PULMONARY EDEMA OR PULMONARY INFLAMMATION

(76) Inventors: Tomas Navratil, Carrboro, NC (US); Kurt E. Brubaker, Durham, NC (US); Jose L. Boyer, Chapel Hill, NC (US); Ward M. Peterson, Morrisville, NC (US); Ramesh Krishnamoorthy, Cary, NC (US)

Correspondence Address: HOWERY LLP, C/O IP DOCKETING DEPARTMENT, 2941 FAIRVIEW PARK DRIVE SUITE 200, FALLS CHURCH, VA, 22042, US

(21) Appl. No.: 12/293,260

(22) PCT Filed: Mar. 16, 2007
(86) PCT No.: PCT/US2007/064234

§ 371 Date: Sep. 16, 2008

Related U.S. Application Data
(60) Provisional application No. 60/783,433, filed on Mar. 17, 2006.

(51) Int. Cl. A61K 9/12; A61K 38/47

(52) U.S. Cl. 424/45; 424/94.61

(57) ABSTRACT

The invention provides a method for preventing or treating pulmonary edema and/or pulmonary inflammation in a subject by the administration of at least one nucleotidase. The subject is preferably a human subject. The nucleotidase is preferably of a mammalian origin, such as a human origin. The NTPDase and NPP families of nucleotidases are particularly useful for the present invention. The method reduces the bioavailability of in situ signaling extracellular nucleotides, thereby disturbing the pathological pathways in ventilator-induced lung injury (VILI) and respiratory syncytial virus infection (RSV) and other disorders such as chronic obstructive pulmonary disorder. Local administration such as inhalation is a preferred route of administration.

### FIELD OF THE INVENTION

This invention relates to a method for preventing or treating pulmonary edema and/or pulmonary inflammation, such as ventilator-induced lung injury and respiratory syncytial virus infection. Specifically, nucleotidases are locally applied into the lung of a subject in need thereof.

### BACKGROUND

### Ventilator-Induced Lung Injury

Mechanical ventilation (MV) is a common and generally effective means of treating a failing lung. Unfortunately, positive-pressure mechanical support can create or contribute to lung injury. Where mechanical ventilation causes or contributes to pulmonary problems, the patient is said to suffer a Ventilator-Induced Lung Injury, or VILI. In light of the fact that more than 280,000 Americans are at risk for VILI each year, and MV support and associated intensive care expenditures are estimated in the billions of dollars, VILI is a major public health concern.

VILI-related mechanisms of injury are not fully understood. However, two mechanisms have been widely studied; alveolar overdistention and pulmonary shear stress.

### Aveolar Overdistention

It is established that mechanical ventilators applying high volumes and pressures can lead to an influx of fluid into the lung. In addition to edema, the injured or ruptured cells trigger a cascade of cellular and biochemical events leading to the inflammation in the lung.

The scientific community has pondered at length whether increased pressure, termed “barotrauma”, or increased volume, termed “volutrauma”, is the underlying cause of VILI. It is currently believed that the pathology of VILI-associated alveolar overdistention is primarily due to stretching alveolar tissue, as opposed to airway pressure itself. Thus, volutrauma, rather than barotrauma, is believed to cause VILI-associated alveolar overdistention.

### Pulmonary Shear Stress

Pulmonary sheer stress can develop due to “volutrauma” as well as due to atelectasis. Atelectasis is a partial or a total collapse of the lung. During atelectasis, the collapsed part of the lung is non-compliant to the ventilation. When an atelectastic lung is ventilated as a whole lung, the non-atelectastic lung portions are subjected to higher sheer. This form of VILI is called atelectrauma. It is associated with ventilation at low end-expiratory lung volumes in injured lungs as well as in normal lungs.

### Biotrauma

VILI is also believed to provoke distal airway and alveolar cell inflammation by increasing the production of proinflammatory cytokines. These released proinflammatory cytokines are capable of entering the general circulation and causing inflammation in other systemic organs. This phenomenon is called biotrauma. It is believed that the inflammation arising during biotrauma is capable of causing multiple organ dysfunction syndrome (MODS). MODS is the progressive impairment of two or more organ systems from an uncontrolled inflammatory response to a severe illness or injury.

Finally, the risk of biotrauma in mechanically ventilated patients is postulated to be heightened in already susceptible patients. This belief is based on experiments showing that previously diseased or injured lungs release more proinflammatory cytokines than healthy lungs, when subjected to subsequent mechanical ventilation.

Respiratory Syncytial Virus infection

The most common cause of lower respiratory tract disease in infants and children worldwide is pneumovirus respiratory syncytial virus (RSV). This disease affects 3.5 to 4 million children patients annually, and hospitalizes approximately 100,000 children annually. Pediatric patient mortality under one year caused by RSV is higher than the one arising from the flu. Newly emerging data shows that RSV is a major cause of death in the elderly as well, killing as many as 15,000 elderly patients a year in the U.S. In short, RSV is a serious public health concern.

A seminal function of airway epithelium is fluid clearance. Normal alveolar epithelial cells maintain proper tissue hydration primarily through an active transport system. Specifically, Na⁺ is transported from the lumen of the lung to the epithelial cells. An osmotic gradient is created by the transepithelial movement of Na⁺, thereby causing passive movement of water from the lumen of the lung to interstitium. This process is termed alveolar fluid clearance (AFC). Proper AFC is essential for respiration because it clears the airway of excessive liquid, while maintaining a thin Epithelial Lining Fluid (ELF) layer, both of which permit efficient gas exchange and normal mucociliary clearance. AFC is decreased when bronchoalveolar epithelium is infected with RSV.

### P2Y and P2X Receptors

Epithelial cell function, including lung epithelium, is modulated by extracellular nucleotide signaling. Of particular importance in the lung are the P2Y₂, P2Y₆ and P2X₇ receptors, which are expressed by airway epithelia. Additional purinergic receptors expressed in the lung are P2X₄, P2Y₁, and A2a receptors. P2Y₂ is an ATP/UTP activated receptor while P2Y₆ is activated by UDP and P2X₇ by ATP. Therefore, released nucleotides and/or the products of their metabolism can stimulate increased intracellular Ca²⁺ through their respective P2Y receptors. Specifically, P2Y₂ receptor binding by ATP or UTP activates phospholipase C, thereby producing inositol-1,4,5-triphosphate (IP₃), which mobilizes Ca²⁺ from internal stores. Stimulation of P2X₇ receptor with ATP induces an ion channel current that increase the influx of calcium into the cell; upon persistent activation by ATP, the P2X₇ receptor turns into a membrane pore.

Usage of nucleotidase has been disclosed in several references as listed below.

Davis, et. al. (Am. J. Physiol. Lung Cell Mol. Physiol. 286:L112-L120 (2004)) reported that in a murine model of RSV infection, alveolar fluid clearance (AFC) was measured over a period of 30 minutes (AFC₃₀). The AFC₃₀ was depressed by 46% on day 2 following the infection of the mice with RSV. Addition of potato apyrase at the start of AFC₃₀ measurements on day 2 post infection completely restored the AFC₃₀ to normal levels.

WO 00/23094 and WO 00/23459 disclose methods for inhibiting platelet activation and recruitment in a mammal comprising administering a soluble CD39 polypeptide.

WO02/071062 discloses that ecto-NTPDase functions on Langerhans cells to counteract the nucleotide inflammatory response caused by chemical irritants.

There are currently no nucleotidase-based treatment methods that target disorders of the lung, let alone VILI and/or RSV specifically. Indeed, there are no pharmacological treatments specifically for VILI and/or RSV at all. In light of the major public health problems associated with these disorders, there is a need for a pharmacological treatment of pulmonary edema and/or pulmonary inflammation.

### PUBLICATIONS

Abbracchio, M. P., and Burnstock, G. 1994. Purinoceptors: are there families of P_{2X} and P_{2Y} purinoceptors? Pharmacol. Ther. 64:445-475.

Amato M B, Barbas C S, Medeiros, D M, et al. Effect of a protective-ventilation strategy on mortality in the acute respiratory distress syndrome. N Engl J Med. 1998;338:347-354. ARDS Network. Ventilation with lower tidal volumes as compared with traditional tidal volumes for acute lung injury and the acute respiratory distress syndrome: the Acute Respiratory Distress Syndrome Network. N Engl J Med. 2000;342:1301-1308.

Barnard, E. A., Burnstock, G., and Webb, T. E. 1994. G protein-coupled receptors for ATP and other nucleotides: a new receptor family. Trends Pharmacol. Sci. 15:67-70.

Buell, G., Collo, G., and Rassendren, F. 1996. P2X receptors: an emerging channel family. Eur. J. Neurosci. 8:2221-2228.

D'Angelo E, Pecchiari M, Baraggia P, Saetta M, Balestro E, and Milic-Emili J. Low-volume ventilation induces peripheral airway injury, and increased airway resistance in normal open-chest rabbits. J Appl Physiol 92: 949-956, 2002.

Darville T and Yamauchi T. Respiratory synctial virus. Pediatr Rev 19:55-61, 1998.

Davis, I. C. et al. 2004. Nucleotide-mediated inhibition of alveolar fluid clearance in BALB/c mice after respiratory syncytial virus infection. AJP Lung. 286:112-120.

Davis, I. C. et al. 2005. Leflunomide prevents alveolar fluid clearance inhibition by syncytial virus infection. Am. J. Respir. Crit. Care Med. Published ahead of print on Dec. 30, 2005 as doi:10.1164/rccm.200508-1200OC.

Dos Santos C C and Slutsky A S. Mechanisms of ventilator-associated lung injury. J Appl Physiol 89: 1645-1655, 2000.

Dreyfuss D, Basset G, Soler P, Saumon G. High inflation pressure pulmonary edema: respective effects of high airway pressure, high tidal volume, and positive end-expiratory pressure. Am Rev Respir Dis. 1988;137:1159-1164.

Dreyfuss D, Basset G, Soler P, Saumon G. Intermittent positive-pressure hyperventilation with high inflation pressures produces pulmonary microvascular injury in rats. Am Rev Respir Dis. 1985; 132:880-884.

Frank J A, Matthay M A. Mechanisms of ventilator-induced lung injury. Crit Care 2003;7:233-241.

Lazarowski, E. R., Paradiso, A. M., Watt, W. C., Harden, T. K., and Boucher, R. C. 1997. UDP activates a mucosal-restricted receptor on human nasal epithelial cells that is distinct from the P2Y2 receptor. Proc. Natl. Acad. Sci. USA. 94:2599-2603.

Matalon S and O'Brodovich H. Sodium channels in alveolar epithelial cells: molecular characterization, biophysical properties, and physiological significance. Annu Rev Physiol 61:627-661, 1999.

Muscedere J G, Mullen J B, Gan K, and Slutsky A S. Tidal ventilation at low airway pressures can augment lung injury. Am J Respir Crit Care Med 149: 1327-1334, 1994.

Nicholas, R. A., Watt, W. C., Lazarowski, E. R., Li, Q., and Harden, T. K. 1996. Uridine nucleotide selectivity of three phospholipase C-activating P2 receptors: identification of a UDP-selective, a UTP-selective, and an ATP- and UTP-specific receptor. Mol. Pharmacol. 50:224-229.

Parker J C, Breen E C, West J F. High vascular and airway pressures increase interstitial protein mRNA expression in isolated rat lungs. J Appl Physiol. 1997;83:1697-1705.

Parker J C, Townsley M I, Rippe B, Taylor A E, Thigpen J. Increased microvascular permeability in dog lungs due to high peak airway pressures. J Appl Physiol. 1984;57:1809-1816.

Parr, C. E. et al. 1994. Cloning and expression of a human P_{2U} nucleotide receptor, a target for cystic fibrosis pharmacotherapy. Proc. Natl. Acad. Sci. USA. 91:3275-3279.

Ricard J D, Dreyfuss D, Saumon G. Production of inflammatory cytokines in ventilator induced lung injury: a reappraisal. Am J Respir Crit Care Med 2001;163:1176-1180.

Tremblay L N and Slutsky A S. Ventilator-induced injury: barotrauma to biotrauma. Proc Assoc Am Physicians 110: 482-488, 1998.

Tremblay L, Valenza F, Riberio S P, Li J, Slutsky AS. Injurious ventilatory strategies increase cytokines and c-fos m-RNA expression in an isolated lung rat model. J Clin Invest 1997;99:944-952.

U.S. Pat. No. 6,335,013 B1—Methods and Materials Relating to CD39-Like Polypeptides

U.S. Pat. No. 6,350,447 B1—Methods and Compositions Relating to CD39-Like Polypeptides and Nucleic Acids

U.S. Pat. No. 6,783,959 B1—Methods and Compositions Relating to CD39-Like Polypeptides and Nucleic Acids

Webb H H, Tierney D F. Experimental pulmonary edema due to intermittent positive pressure ventilation with high inflation pressures: protection by positive end-expiratory pressure. Am Rev Respir Dis. 1974;110:556-565.

WO 00/23094—Methods of Inhibiting Platelet Activation and Recruitment

WO 00/23459—Inhibitors of Platelet Activation and Recruitment

WO 02/071062 A2—Diagnosis and Treatment of Inflammation and Hyperactive Immune Conditions

Zimmermann H (2000) Extracellular metabolism of ATP and other nucleotides. Naunyn Schmiedeberg's Arch Pharmacol 362: 299-309

### SUMMARY OF THE INVENTION

The present invention is directed to a method for preventing or treating pulmonary edema. The present invention is also directed to a method for preventing or treating pulmonary inflammation. The method comprises administering to a subject in need thereof at least one nucleotidase. In one embodiment, the subject is a human subject. In another embodiment, the nucleotidase is not a potato apyrase. The nucleotidase is preferably of a mammalian origin, such as a human origin. The NTPDase and NPP families of nucleotidases are particularly useful for the present invention. The preferred ectonucleotidases are NTPDase 3, NTPDase 1, NPP3 and NPP1, most preferably the forms of the protein that lacks of the membrane anchoring domains but conserves the catalytic domains important for enzymatic activity.

The present invention is useful for treating ventilator-induced lung injury, respiratory syncytial virus infection, chronic bronchitis, and chronic obstructive pulmonary disease. The nucleotidase can be administered systemically or locally, with local administration being preferred. Local administration such as liquid instillation, inhalation of aerosolized solution via nebulizer or a metered dose pressurized inhalation fluid preparation, inhalation of dry powder by inhaler, and directing soluble or dried material of a desired particle size distribution into the air stream during mechanical ventilation, are preferred routes of administration.

### BRIEF DESCRIPTION OF THE DRAWINGS

FIG. 1 shows the stability comparison of potato apyrase and human NTPDase 3 in cerebrospinal fluid.

### DETAILED DESCRIPTION

The inventors have discovered that administering one or more nucleotidases into the lungs of patients is useful in preventing or reducing pulmonary edema and/or inflammation. The inventors have discovered that local application of nucleotidases to lung epithelium is especially beneficial. The present invention provides a method of preventing or treating pulmonary edema and/or pulmonary inflammation.

### Definitions

The term “edema” refers to an abnormal accumulation of extra-vascular fluid. Of particular relevance here is “pulmonary edema” which refers specifically to fluid accumulation within the lung interstitium or the lumen of the lung. Pulmonary edema is associated with a variety of systemic or lung diseases including respiratory syncytial virus infection (RSV), human matapneumovirus, pneumonia, influenza, ventilator induced lung injury (VILI), acute respiratory distress syndrome (ARDS), acute lung injury (ALI), and chronic obstructive pulmonary disease (COPD) such as chronic bronchitis and emphysema.

“Extracellular nucleotide” is any nucleotide or nucleotide-containing molecule which is found outside a cell. Extracellular nucleotides of particular importance in the present invention are mononucleotides such as ATP, UTP, ADP, UDP, AMP, and UMP; dinucleotides such as Ap₄A, Ap₅A, Up₄U, Ap₄U, Ap₅U; nucleotide sugars such as UDP-glucose and nucleoside entities. These substances are present in the liquid that covers airway surfaces, and they mediate important physiological events through interaction with P2 and P1 nucleotide and nucleoside receptors, respectively.

“Inflammation” generally refers to a localized reaction of tissue, characterized by the influx of immune cells, which occurs in reaction to injury or infection. Specifically, “pulmonary inflammation” is characterized by migration of inflammatory cells into the interstitium and the lumen of the lung, release of pro-inflammatory cytokines and chemokines, lung tissue remodeling and lung tissue apoptosis or necrosis. Pulmonary inflammation accompanies a variety of systemic or lung diseases including those noted in the aforementioned pulmonary edema definition.

“Local administration” is any method of applying the medicament directly, as opposed to systemically, to the desired site. Local administration can be achieved by any method which results in delivery of nucleotidase to the epithelium of the lung.

“Non-soluble nucleotidase”, as used herein, is a nucleotidase that is not able to be suspended in the aqueous layer covering the lung. The non-soluble nucleotidase in general contains a transmembrane spanning region, or is attached to a membrane by a post translational modification such as GPI anchoring or lipid modification.

“Nucleotidase,” as used herein, is any hydrolase enzyme that is able to (a) catalyze the hydrolysis of a nucleotide to yield a nucleoside and ortho- and/or pyro-phosphate, (b) catalyze the hydrolysis of a nucleotide to yield a nucleoside monophosphate and ortho- and/or pyro-phosphate, (c) catalyze the hydrolysis of a nucleotide to yield nucleotide diphosphate and ortho- and/or pyro-phosphate, (d) catalyze the hydrolysis of a dinucleotides to mononucleotide and the remainder of the substrate moiety, which may be further hydrolyzed, or (e) catalyze the hydrolysis of a nucleotide-sugar conjugate (e.g. UDP-glucose) to yield a nucleoside or a nucleoside monophosphate and the remainder of the substrate moiety, which may be further hydrolyzed. Nucleotidases of particular importance in the present invention include those from the NTPDase and NPP families.

“Nucleotide” refers to a compound consisting of a nucleoside (sugar plus purine or pyrimidine base) and at least one phosphate group.

The terms “signaling” or “cellular signaling” refer the interaction between one or more extracellular constituents and one or more cellular receptor molecules, where that interaction effectuates stimulation or inhibition of a cellular function or activity. For example, the extracellular constituents of ATP and its metabolites, collectively called ligands, bind to P2 receptors of epithelial cells. The ligand/receptor interaction causes cellular responses such as transportation of ions, or activation of intracellular enzymes, ultimately leading to changes in localized fluid accumulation.

“Soluble nucleotidase”, as used herein, is a nucleotidase that has the ability to be suspended in the aqueous layer covering the airways.

A “therapeutically effective dose” is the amount of therapeutic material or pharmaceutical composition sufficient to treat, heal, prevent or ameliorate the pathological condition. The therapeutically effective dose varies by species, size of patient, severity of pathology and other factors. The therapeutically effective dose is defined in terms of improving at least one of the parameters relevant to measurement of pulmonary edema and/or pulmonary inflammation.

### Definition of Enzyme Classes and Accession of Enzyme DNA Sequences

Based on the bioinformatics tools described here, one can uniquely and unambiguously identify every DNA sequence of a gene coding for a polypeptide that belongs to a particular enzyme class. This would be known to the person of ordinary skill in the art. Currently, the definitions of enzyme classes are governed by the Nomenclature Committee of the International Union of Biochemistry and Molecular Biology. The general classification of enzymes is based on their enzymatic activity and the type of substrate. The general classes and subclasses of enzymes are accessible via a variety of interfaces. The enzymes of this invention are referenced by the name of their class, which is available via the Enzyme Date Bank (Bairoch A.), the ENZYME database described in Nucleic Acids Res 28:304-305 (2000), and is accessable via ExPASy interface, a database maintained by the Swiss Institute of Bioinformatics (see Gasteiger E., et al. ExPASy: the proteomics server for in-depth protein knowledge and analysis, Nucleic Acids Res. 31:3784-3788 (2003)); also can be found at website address us.expasy.org/cgi-bin/enzyme-search-cl. Via this interface, each member of a given class of enzymes can be discreetly identified based on the unique GenBank accession number. The GenBank accession number points to the unique DNA sequence coding for a protein of interest. GenBank DNA database is maintained by the National Center for Bioinformatics as a recognized body supported by the National Institute of Health (see Dennis A., et al. GenBank, Nucl. Acids Res. 35: D21-D25 (2007); doi:10.1093/nar/gkl986).

If the location of the sequence databases change, the person of ordinary skill in the art would know where to find the database's new location, or an alternative source for the sequence.

The present invention provides a method for preventing or treating pulmonary edema and/or pulmonary inflammation in a human subject. The method comprises the steps of: (a) identifying a human subject who is at risk of developing edema and/or pulmonary inflammation, or who is suffering from pulmonary edema and/or pulmonary inflammation; (b) administering to said human subject at least one nucleotidase; and (c) evaluating the treatment of said human subject.

The present invention also provides a method of treating pulmonary edema and/or pulmonary inflammation in a subject including human subjects and non-human subjects such as mice and rats. The method comprises the steps of: (a) identifying a subject who is suffering from pulmonary edema and/or pulmonary inflammation; and (b) administering to said subject at least one nucleotidase; wherein said nucleotidase is not potato apyrase.

In the present method, the administration of nucleotidase degrades extracellular nucleotides such as ATP, UTP and UDP, which activate P2Y₂, P2Y₆ and P2X₇ receptors. Thus, the administration of nucleotidase acts similarly as administration of antagonists of P2Y₂, P2Y₆ and P2X₇ receptors.

The present invention provides a method of preventing or treating a variety of pulmonary diseases. The widespread applicability of the method lies in the fact that the method targets the underlying cause of the pathologies; i.e., increased levels of extracellular nucleotides and its functional consequences. Extracellular nucleotides are released when lung tissue is subjected to pressure, shear forces, injury or infection or other exacerbations. These increased extracellular nucleotides act on the receptors, such as P2Y₂, P2Y₆ and P2X₇ receptors, found in airway epithelium and inflammatory cells. The receptor binding activates a series of bioprocesses such as increased Cl⁻ secretion which is followed by water efflux from the airway epithelial cells into the lumen of the lung, and increased mucin secretion from goblet cells into the lumen of the lung. The bioprocesses ultimately result in pulmonary edema and/or inflammation. For example, in RSV, the infected bronchoalveolar epithelium releases UTP, ATP and potentially other nucleotides, resulting in increased extracellular levels of these nucleotides. The released nucleotides interact with the P2Y and P2X receptors in the airway epithelium and disrupt Na⁺ transport. The Na⁺ transport disruption contributes to decreased alveolar fluid clearance.

Pathological effects of pulmonary edema and/or pulmonary inflammation include functional or structural abnormalities arising at the cellular, tissue, organ and/or system level, due to edema and/or inflammation of the lung or lung-related structures. Pathological effects of pulmonary edema and/or pulmonary inflammation, by definition, decrease the respiratory function of the lung. Symptoms of pathological effects include wheezing, coughing and labored breathing or hypoxia, respiratory distress and necessity for mechanical ventilation.

The present invention is suitable to prevent or treat respiratory syncytial virus infection (RSV), human matapneumovirus, pneumonia, influenza, ventilator induced lung injury (VILI), acute respiratory distress syndrome (ARDS), acute lung injury (ALI), chronic obstructive pulmonary disease (COPD). The present invention is particularly suitable to prevent or treat VILI, RSV and other disorders such as COPD.

### Nucleotidases

Any nucleotidase that has the capability to hydrolyze extracellular nucleotides, dinucleotides or nucleotide-sugar conjugates, has a proper stability in a biological fluid, and does not exhibit an unfavorable therapeutic profile, is appropriate for the invention at issue.

Nucleotidase of a mammalian origin, particularly a human origin, is preferred for the present invention. Non-human nucleotidases, especially non-mammalian nucleotidases, when used in human patients, often present risks for increased inflammation due to immunogenicity. In one embodiment, the nucleotidase is glycosylated or pegylated to increase its in vivo stability, for example, from being degraded by proteases. Potato apyrase in general is not a desirable nucleotidase for the present invention because of its instability in a biological fluid and its non-human origin.

Nucleotidases suitable for this invention include soluble nucleotidases and non-soluble nucleotidases. Some nucleotidases are naturally soluble (devoid of membrane-spanning regions), and some are naturally non-soluble (attached to the membrane). There are also nucleotidases that naturally exist in both soluble and non-soluble forms, due to the processing of the enzyme or a protease cleavage of the enzyme.

In one embodiment, the nucleotidase used in this invention is soluble. Such nucleotidase has the ability of being suspended in the aqueous layer covering the lung, thus it reaches the site of action effectively and provides a therapeutic effect. The soluble nucleotidase either does not have the transmembrane region, or is attached to a vesicle that makes the nucleotidase soluble. For example, the vesicle can be membraneous vesicle with the nucleotidase attached to the vesicle facing the extracellular space. The methods used to achieve soluble nucleotidase include, but are not limited to, preparing constructs devoid of trans-membrane regions, preparing site-directed mutagenesis variants, preparing deletion or insertion variants, preparing fusion constructs chimeric forms of the protein or other conjugates, and solubilizing nucleotidase by detergent. In one embodiment, the soluble nucleotidase is native. In one embodiment, the soluble nucleotidase is a native enzyme. In another embodiment, the soluble nucleotidase is prepared by recombinant technology.

In another embodiment, the nucleotidase used in this invention is non-soluble. The non-soluble nucleotidase either has the transmembrane spanning region and is capable of inserting itself into the surface of the lung epithelium, or it is contained in lipid vesicles that fuse with the lung epithelium, or it attaches to a surface marker on the lung epithelium via an antibody or ligand that is engineered to be a part of the nucleotidase. When a particle is introduced into the lung, the mucociliary clearance system starts moving the particle from distal to proximal lung and then to expectoration. When a non-soluble nucleotidase attaches to the surface of the lung epithelium where it lands, it has the advantage of having a longer duration of action because of the slower clearance from the lung.

Nucleotidases useful for this invention include ecto-nucleoside triphosphate diphosphohydrolase (NTPDase) families and ecto-nucleotide pyrophosphatase/phosphodiesterase (NPP) families. The NTPDase family hydrolyzes nucleoside 5′-triphosphates and nucleoside 5′-diphosphates. The NPP family of enzymes hydrolyzes nucleoside 5′-triphosphates and nucleoside 5′-diphosphates, both di- and mononucleotides and nucleotide-sugar conjugates.

NTPDase includes NTPDase 1, NTPDase 2, NTPDase 3, and NTPDase 8. NTPDase 1 (ecto-ATPDase, CD39, apyrase) hydrolyzes both nucleoside triphosphates and nucleoside diphosphates. NTPDase 2 (ecto-ATPase, CD39L1) preferentially hydrolyzes nucleoside triphosphates. NTPDase 3 (ectoATPDase, CD39L3) hydrolyzes both nucleoside triphosphates and nucleoside diphosphates with a 3:1 preference for the triphosphates. NTPDase8 hydrolyzes both nucleoside triphosphates and nucleoside diphosphates with a 3:1 preference for the triphosphates. These four isozymes are integral proteins of the plasma membrane. They contain short cytosolic N- and C-termini, and large extracellular domains containing apyrase conserved regions and conserved catalytic regions. Other NTPDase family proteins include NTPDase 4, NTPDase 5 (CD39L2), and NTPDase 6 (CD39L4). The NTPDase families include all three forms of enzymes: soluble, non-soluble (membrane-attached), and both forms (soluble and non-soluble). NTPDase 5 and 6 are both soluble and non-soluble. The rest of the NTPDase enzymes are naturally in its membrane-attached form. Soluble NTPDase 1 and soluble NTPDase 3 are particularly useful for the present invention. Soluble NTPDase 1 and soluble NTPDase 3 for example can be prepared by recombinant technique.

NPP family comprises three polypeptides encoded by different genes with predicted short cytosolic N-termini, one transmembrane domain, and a long extracellular domain bearing the catalytic site. This group of isoenzymes includes NPP1 (PC-1), NPP2 (autotaxin, PD-Iα), and NPP3 (PD-1β, B10). NPP isoenzymes display broad substrate specificity for hydrolyzing nucleoside 5′-triphosphate (NTP) to nucleoside monophosphate (NMP) plus pyrophosphate, nucleoside 5′-diphosphate (NDP) to NMP plus orthophosphate, dinucleoside polyphosphates to NPₙ₋₁ plus NMP, nucleotide-sugar conjugates such as UDP-glucose and cAMP to AMP. NPP isoenzymes also hydrolyze nucleic acids, NAD, and nucleotide-sugar conjugates. The NPP family enzymes include all three forms of enzymes soluble; non-soluble (membrane-attached), and both forms (soluble and non-soluble). In vitro, all NPP enzymes have been shown to exist in both forms. In vivo, only NPP2 has been shown to exist in both forms. Some of the NPP family enzymes exist in vivo in their soluble form that is detached from the cellular membrane.

Particular nucleotidases suitable for the present invention include those that possess the capability to hydrolyze extracellular nucleoside mono or polyphosphates. This group includes the following classes of enzymes as defined in the above-referenced enzyme databases based on the recommendations of the Nomenclature Committee of the International Union of Biochemistry and Molecular Biology

(www.chem.qmul.ac.uk/iubmb/nomenclature/): adenosinetriphosphatase, apyrase, nucleoside-diphosphatase, ATP diphosphatase, nucleotide diphosphatase, ADP-ribose diphosphatase, nucleoside-triphosphatase, nucleoside-triphosphate diphosphatase, ADP-sugar diphosphatase, triphosphatase, thymidine-triphosphatase, UDP-sugar diphosphatase, phospholipid-translocating ATPase, magnesium-importing ATPase, cadmium-exporting ATPase, copper-exporting ATPase, zinc-exporting ATPase, proton-exporting ATPase, sodium-exporting ATPase, calcium-transporting ATPase, sodium/potassium-exchanging ATPase, hydrogen/potassium-exchanging ATPase, chloride-transporting ATPase, potassium-transporting ATPase, H(+)-transporting two-sector ATPase, sodium-transporting two-sector ATPase, arsenite-transporting ATPase, monosaccharide-transporting ATPase, oligosaccharide-transporting ATPase, maltose-transporting ATPase, glycerol-3-phosphate-transporting ATPase, polar-amino-acid-transporting ATPase, nonpolar-amino-acid-transporting ATPase, oligopeptide-transporting ATPase, nickel-transporting ATPase, sulfate-transporting ATPase, nitrate-transporting ATPase, phosphate-transporting ATPase, phosphonate-transporting ATPase, molybdate-transporting ATPase, Fe(3+)-transporting ATPase, polyamine-transporting ATPase, quaternary-amine-transporting ATPase, vitamin B12-transporting ATPase, iron-chelate-transporting ATPase, manganese-transporting ATPase, taurine-transporting ATPase, guanine-transporting ATPase, capsular-polysaccharide-transporting ATPase, lipopolysaccharide-transporting ATPase, teichoic-acid-transporting ATPase, heme-transporting ATPase, beta-glucan-transporting ATPase, peptide-transporting ATPase, xenobiotic-transporting ATPase, steroid-transporting ATPase, cadmium-transporting ATPase, fatty-acyl-CoA-transporting ATPase, alpha-factor-transporting ATPase, channel-conductance-controlling ATPase, protein-secreting ATPase, mitochondrial protein-transporting ATPase, chloroplast protein-transporting ATPase, Ag(+)-exporting ATPase, myosin ATPase, dynein ATPase, microtubule-severing ATPase, plus-end-directed kinesin ATPase, minus-end-directed kinesin ATPase, vesicle-fusing ATPase, peroxisome-assembly ATPase, proteasome ATPase, chaperonin ATPase, non-chaperonin molecular chaperone ATPase and nucleoplasmin ATPase.

The nucleotidase of the present invention includes native enzymes, and those made by the recombinant technique. For Example, the nucleotidase of the present invention include NTPDase-like proteins disclosed in Chadwick et al (Genomics 50: 357-367 (1998)); U.S. Pat. Nos. 6,335,013; 6,350,447; 6,783,959; WO00/23459; the sequences of those CD39-like proteins are incorporated herein by references. The nucleotidase of the present invention also includes humanized nucleotidase. Humanized nucleotidase is a nucleotidase from another organism not present in human but made more human-like from engineered construct.

The nucleotidase, as used herein, includes the whole enzyme polypeptide, a fragment thereof, and a variant thereof, which exhibits the nucleotidases activity. In one embodiment, the fragment or variant exhibits at least about 70% identical in amino acid sequence to the amino acid sequence of native enzyme. In more preferred embodiments, the fragment or variant exhibits at least about 80%, 90%, 95%, 98%, or 99% identical in amino acid sequence to the amino acid sequence of native nucleotidase. Percent identity, in the case of both polypeptides and nucleic acids, can be determined by visual inspection. Percent identity can also be determined using the alignment method of Needleman and Wunsch (J. Mol. Biol. 48:443, 1970) as revised by Smith and Waterman (Adv. Appl. Math 2:482, 1981). Preferably, percent identity is determined by using a computer program, for example, the GAP computer program version 10.× available from the Genetics Computer Group (GCG; Madison, Wis., see also Devereux et al., Nucl. Acids Res. 12:387, 1984). The preferred default parameters for the GAP program include: (1) a unary comparison matrix (containing a value of 1 for identities and 0 for non-identities) for nucleotides, and the weighted comparison matrix of Gribskov and Burgess, Nucl. Acids Res. 14:6745, 1986, as described by Schwartz and Dayhoff, eds., Atlas of Protein Sequence and Structure, National Biomedical Research Foundation, pp. 353-358, 1979 for amino acids; (2) a penalty of 30 (amino acids) or 50 (nucleotides) for each gap and an additional 1 (amino acids) or 3 (nucleotides) penalty for each symbol in each gap; (3) no penalty for end gaps; and (4) no maximum penalty for long gaps.

Other programs used by one skilled in the art of sequence comparison can also be used. For fragments of nucleotidase, the percent identity is calculated based on that portion of nucleotidase that is present in the fragment.

### Therapeutic Protocol

A therapeutic effective amount of at least one nucleotidase is administered to a patient in need of such treatment. The patient either already has the symptoms of pulmonary edema and/or inflammation, or is identified as having risk of pulmonary edema and/or inflammation. The nucleotidase is administered at a frequency that achieves desired efficacy. What constitutes desired efficacy is determined by a physician or other authorized health-care professional. Whether or not sufficient efficacy has been reached is determined by subjective and/or objective indicia of efficacy. In general, the nucleotidase is administered into the lumen of the lung of patients in the amounts ranging from 0.01 Unit to 100,000 Units. One Unit of enzyme is defined as an amount of enzyme that liberates 1.0 μmole of inorganic phosphate from adenosine 5′triphosphate per minute at 30° C. After initial dose, additional doses are optionally administered.

“Subjective indicia of therapy toleration” refers to how the therapeutic material is perceived to be tolerated by the patient or physician. The therapeutic material is considered subjectively tolerated well if, for example, the patient reports no ill effects believed to be associated with administration of the therapeutic material.

“Objective indicia of efficacy” include demonstrable improvement in measurable signs of edema and/or inflammation. Such signs of improvement include increased blood/oxygen saturation or decreased frequency of coughing and/or wheezing, improved forced expiratory volume (FEV₁), decreased mortality or morbidity, decreased length of hospital stay, decreased need for mechanical ventilation, lower amount of inflammatory cells infiltrating the lung, lower levels of proinflammatory cytokines and chemokines, improved alveolar fluid clearance rate, and/or smaller pulmonary edema, as determined by any radiographic or other detection method.

“Parameters for measurement of pulmonary edema and/or pulmonary inflammation” include depression of respiratory function, amount of epithelial lining fluid, wet to dry lung weight, alveolar fluid clearance, radiographic visualization methods, level of oxygenation/hypoxia, FEV₁, FVC₁, length of a hospital stay, mortality or morbidity, increase in general quality of life, requirement for mechanical ventilation, the levels of inflammatory cells in the lung or outside of the lung in other anatomical compartments or spaces including systemic circulation, the amount of pro-inflammatory molecules including cytokines and chemokines in the lung or outside of the lung in other anatomical compartments or spaces including systemic circulation, pathological remodeling of the airway, patient-reported or physician-observed signs such as ease of breathing, or severity of coughing and/or wheezing, and feelings of well-being.

### Routes of Administration

The key of the invention lies in the ability of the nucleotidase to hydrolyze the in situ nucleotides in the lung. Any method of delivering the nucleotidase to the lumen of the lung, including local administration and systemic administration, is suitable for the present invention. Systemic administration is introducing a medicament into the circulation. Examples of systemic administration include oral ingestion, or intravenous or subcutaneous or intraperitoneal or intrathecal or intramuscular administration.

A preferred embodiment of the invention is localized administration. Local administration includes inhalation, topical application or targeted drug delivery. Methods of inhalation include liquid instillation, instillation as a pressurized fluid preparation via metered dose inhaler or equivalent, or inhalation of an aerosolized solution via nebulizer (preferred), inhalation of dry powder (more preferred), and directing soluble or dried material into the air stream during mechanical ventilation (also more preferred).

An example of targeted drug delivery is enclosure of the nucleotidase within a liposome, where the liposome is coated with a specific antibody whose antigen is expressed in the targeted lung tissue.

It can be advantageous to construe a controlled delivery system of the nucleotidases since such an inhaled product targets the site of action, presents the compound of interest in small regimented quantities and reduces/minimizes any unwanted side effects. There are some limitations to the effectiveness of controlled release delivery systems since prolonged residence of some of the materials in the lungs can pose long term toxicity problems.

Yet another example of a delivery system can include nanoparticulates or microparticulates compositions of the nucleotidase. In such a case, the nucleotidase is formulated as a nanosuspension wherein the carrier is loaded with the nucleotidase; such a preparation is then filtered through a fine porous membrane or suitable filtering medium or is exposed to solvent interchanges to produce nanoparticles. Such nanoparticulate preparations can be freeze dried or held in suspension in an aqueous or physiologically compatible medium. The preparation so obtained can be inhaled by suitable means.

Another example of a suitable preparation includes a reconstitutable preparation. In this case, the nucleotidase is formulated in a preparation to contain the necessary adjuvant to make it physiologically compatible. Such a preparation can be reconstituted by addition of water for injection or suitable physiological fluids, admixed by simple agitation and inhaled using appropriate techniques described above.

The nucleotidases described above can also be prepared into dry powder or equivalent inhalation powders using the well known art of super critical fluid technology. In such a case, the nucleotidase is admixed with appropriate excipients and milled into a homogenous mass using suitable solvents or adjuvants. Following this, this mass is subjected to mixing using super critical fluid technology and suitable particle size distribution achieved. The particles in the formulation need to be of a desired particle size range such that the particles can be directly inhaled into the lungs using a suitable inhalation technique or introduced into the lungs via a mechanical ventilator. Alternatively, a formulation can be designed such that the particles are large enough in size thereby offering sufficient surface area to dissolve completely in a suitable fluid when admixed together or to dissolve sufficiently enough prior to nebulization into the lungs.

In an attempt to prevent particle size growth and minimize crystal growth of the nucleotidases, one embodiment is to include the use of spray-dried particles that have better aerodynamic properties than micronized material. This can be further extended to coat the surface of the hydrophilic molecule with one or more layers of hydrophobic material.

Another embodiment of this composition would involve the preparation of freeze-dried or lyophilized preparation of the nucleotidases. Such a preparation would be made to protect the inherent instability of the molecule due to physical or chemical changes induced in the presence of certain solvents or processing techniques. To this end, the use of cryoprotectants to further maintain the tertiary structure of the proteins is contemplated. The lyophilized preparations can be used as is in the form of a dry powder inhaler or be admixed with other suitable adjuvants to be used as dry powder inhaler or as a nebulized preparation.

### Pharmaceutical Formulations

The nucleotidase can be administrated to the patient with any other components that act in synergy with, or otherwise improve the tolerability and or efficacy of the nucleotidase. Examples include potentiators, complexing agents, targeting agents, stabilizing agents, cosolvents, pressurized gases, or solubilizing conjugates. The nucleotidase can be formulated in a pharmaceutical formulation comprising at least one nucleotidase and a pharmaceutically acceptable carrier, such as excipients, diluents, fillers, salts, buffers, stabilizers, solubilizers and other materials known in the art. In addition, the nucleotidase can be administrated to the patient in combination with other drugs.

Pharmaceutically acceptable carriers include solvents (e.g saline solution), dispersion media, and absorption delaying agents. Other agents could include coatings, anti-infectives including antibacterial and antifungal agents, isotonic and absorption delaying agents, and the like that are physiologically compatible. The carrier can be suitable for inhalation, intravenous, intramuscular, subcutaneous, or topical administration (e.g., by injection or infusion). Depending on the route of administration, the nucleotidase can be coated in a material to protect the compound from the action conditions that can inactivate the compound. Various pharmaceutically acceptable carriers, which are physically and chemically compatible, and which do not mitigate the biological activity of these potent nucleotidases, are desirable. Desirable carriers for dry powders include sugars, modified sugars, cellulosic derivatives, and gelatin.

Acceptable excipients include sugars such as lactose, sucrose, mannitol, or sorbitol; cellulose preparations such as, for example, maize starch, wheat starch, rice starch, potato starch, gelatin, gum tragacanth, methyl cellulose, hydroxypropylmethylcellulose, sodium caroxymethylcellulose, and/or polyvinylpyrrolidone (PVP). Preferred excipients include lactose, gelatin, sodium carboxymethyl cellulose, and low molecular weight starch products.

Acceptable suspending agents that can also serve as valve lubricants in pressurized pack inhaler systems are desirable. Such agents include oleic acid, simple carboxylic acid derivatives, and sorbitan trioleate.

Acceptable diluents include water, saline, phosphate-buffered citrate or saline solution, and mucolytic preparations. Other suitable diluents include alcohol, propylene glycol, and ethanol; these solvents or diluents are more common in oral aerosol formulations. Physiologically acceptable diluents that have a tonicity and pH compatible with the alveolar apparatus are desirable. Preferred diluents include isotonic saline, phosphate buffered isotonic solutions whose tonicity have been adjusted with sodium chloride or sucrose or dextrose or mannitol.

Acceptable fillers include glycerin, propylene glycol, ethanol in liquid or fluid preparations. Suitable fillers for dry powder inhalation systems include lactose, sucrose, dextrose, suitable amino acids, and derivatives of lactose. Preferred fillers include glycerin, propylene glycol, lactose and certain amino acids.

Acceptable salts include those that are physiologically compatible and provide the desired tonicity adjustment. Monovalent and divalent salts of strong or weak acids are desirable. Preferred salts include sodium chloride, sodium citrate, ascorbates, and sodium phosphates.

Acceptable buffers include phosphate or citrate buffers or mixed buffer systems of low buffering capacity. Preferred buffers include phosphate or citrate buffers.

Acceptable coating agents to provide a hydrophobic sheath around the hydrophilic cores can include caproic and lauric acids. During the preparation of liposomes, the use of diphosphatidyl choline or diphosphatidyl myristyl choline or other suitable materials can be included to provide protection to the molecules or formulation.

Acceptable stabilizers include those that provide chemical protection to the molecule of interest or those that enhance the overall physical stability of the final preparations. Acceptable stabilizers do not inhibit the nucleotidase activity. Such stabilizers include antioxidants such a sodium metabisulfite, benzalkonium chloride, cetylpyridinum chloride, alcohol, polyethylene glycols, butylated hydroxyanisole, butylated hydroxytoluene, disodium edetate. The preferred stabilizers include sodium metabisulfite, disodium edetate and polyethylene glycols. Included within this class of stabilizers would be cryoprotectants such as polyethylene glycols, sugars, and carrageenans.

Acceptable solubilizers include propylene glycol, glycerin, suitable amino acids, complexing agents such as cyclodextrins, sorbitol solution, or alcohol. Solubilizers including ethanol, propylene glycol, glycerin, sorbitol, and cyclodetrins are desirable. The preferred solubilizers include propylene glycol, sorbitol, and cyclodextrins.

Other therapeutics which can be used with the inventive therapeutic material, for purposes of creating a “drug cocktail” include (i.e. anti-infective, proteolytics, mucolytics, bronchodilators, anti-inflammatory materials, corticosteroids, surface proteins normally expressed in the lung, DNAses, inhibitiors of protease activity, metalloprotease inhibitors, materials that induce lactation, induce somnia, or control respiratory distress, etc). In the preferred embodiment anti-infectives including antibacterial agents, antiviral agents, antifungal agents, bronchodilators, and possibly long acting corticosteroids would be combined with the therapeutic material.

Therapeutic material can be formulated for inhalation with use of a suitable propellant such as dichlorodifluoromethane, dichloroflouromethane, dichlorotetrafluoroethane, carbon dioxide or other gas. Preferred propellants include non-CFC related class of propellants or related analogs.

The pharmaceutical material can also be dried into an inhalable dry powder. This can be achieved by mixing with suitable adjuvants that are compatible with the nucleotidase and offer biological compatibility; such mixtures can be suitably communited and reduced in particle size distribution by milling or spray drying or super critical fluid processing. A desirable method of drying the pharmaceutical material for inhalation is spray drying or conventional bed drying or super critical fluid processing. A preferred method is spray drying or super critical fluid processing techniques.

### Packaging

The therapeutic material can be packaged so as to allow a practitioner to formulate it into pharmaceutical compositions as needed. Alternatively, the pharmaceutical composition, itself, can be packaged, thereby requiring de minimus formulation by practitioner. In any event, the packaging should maintain efficacy and chemical and aesthetic integrity of therapeutic material or pharmaceutical composition to the extent reasonably possible.

Possible methods of packaging include blister packaging, packaging in unit dose vials, packaging in blow-fill-seal plastic vials, filling in pressurized canisters, packaging in a two compartment system wherein the contents of the two compartments are admixed by mechanical agitation prior to administration and the contents used within a specified period of time. Packaging the material by filling into a plastic vial whose contents can be easily opened, contents dispensed, and empty container disposed off to prevent re-use or contamination is deemed advantageous. The most preferred packaging method is packaging the formulation in a blister packaging system wherein the contents are held protected from heat, light, and other environmental extremes.

Where the therapeutic material is packaged for inhalation, the pharmaceutical composition can be packaged in aerosol spray canister or packaged for use with nebulizer or ventilator. This can be achieved by directly filling the container using the common techniques of cold filling or filling under a pressurized system or simply filling the product formulation under gravity feed in an aseptic environment. Depending on the nature of the final formulation, this can be achieved by a cold-filling technique wherein the composition is packaged in an aerosol canister under high pressure in a clean-room environment, preferably under aseptic conditions. Alternatively, if the composition is a simple solution, homogenous fluid, or well-mixed suspension product, the formulation can be filled into unit dose blow-fill-seal vials under a gravity feed or filled into blister packs wherein the formulation is filled into unit cavities and secured close with suitable foil or equivalent packaging to protect it from environmental extremes. This operation is preferred to be carried out under aseptic conditions, preferably under ambient or sub-ambient temperatures with little to no environmental extremes. It is desirable that such filling and packaging operations be conducted in relatively particulate free environments with minimal microbiological loads (especially absence of Pseudomonas and other similar pathogens) and be done with minimal exposure to direct human interface. The blister packaging can be done most optimally with cold fill packaging. The product compositions can be directly filled into the final container of choice by direct metered transfer (either gravimetrically or volumetrically) and secured close with appropriate closure systems.

The invention is illustrated further by the following examples that are not to be construed as limiting the invention in scope to the specific procedures described in them. Without further elaboration, it is believed that one skilled in the art can, using the preceding description, utilize the present invention to its fullest extent. The following preferred specific embodiments therefore are to be construed as merely illustrative, and not limited of the remainder of the disclosure in any way whatsoever.

### EXAMPLES

### Example 1

### Expression and Purification of Human NTPDase 3

Human NTPDase 3 (CD39L3) sequence is shown in Chadwick et al (Genomics 50: 357-367 (1998)). Partial NTPDase 3 sequence of human origin was cloned into a eukaryotic expression vector designed for high-level expression and secretion in mammalian hosts. The partial NTPDase 3 sequence did not include the trans-membrane regions of NTPDase 3, thus making the protein soluble. In this expression vector, NTPDase 3 construct sequence was cloned in in-frame with a sequence cloning for a peptide containing c-myc epitope and six tandem histidine residues for detection and purification of the expressed fusion protein. This soluble human recombinant shrNTPDase 3 construct was designated as NTPDase 3.

The shrNTPDase 3 expression vector was transfected into an immortalized CHO cell line, and stable cell line expressing shrNTPDase 3 construct was prepared. The cells were grown in New Brunswick Scientific, Inc. Spinner Basket with Fibra-Cel Support. The Fibra-Cel disks are a solid support material for the growth of animal and plant cells. The disks are used in the packed-bed basket of the NBS cell culture spinner flasks. The disks are used as a matrix for the growth of cells when secreted products are desired.

The shrNTPDase 3 CHO cells were grown in F12 media with 10% FBS in the spinner flasks. The media was changed every 48 hours until maximum production of shrNTPDase 3 was achieved as monitored by ATPase activity measurements via malachite green assay. Then the spinner flask was changed into a ProCho-4 protein free media (Cambrex, Inc.). The ProCho-4 protein free media containing secreted shrNTPDase 3 was collected every 48 hours (400 ml/spinner flask/48 hours). The 400 ml of the media was reduced to 50 to 100 ml by concentration in Millipore filtration cell and dialyzed against 500 mM NaCl, 50 mM Tris-HCl pH 7.4. The dialyzed sample was loaded onto GE HisTrap HP 5 ml FPLC column and purified on Akta 10 FPLC instrument (General Electrics). The His-tagged soluble human recombinant NTPDase 3 was eluted from the column by 25 column volume immidazole gradient and collected by fractionation.

The fractions were pooled according to the ATPase activity as determined by malachite green assay. The purity of the pooled fractions was determined by sodium dodecyl sulfate polyacryamide gel electrophoresis (SDS-PAGE) and subsequent silver stain of the SDS-PAGE gel.

### Example 2

### Attenuation of Pulmonary Edema or Inflammation in Human Patients

Patients suffering from pulmonary edema and/or the inflammation are administered with NTPDase 3 (CD39L3), into the lumen of their lung. Human recombinant nucleotidase NTPDase 3 was manipulated to express and purify as a soluble nucleotidase (Example 1). This soluble nucleotidase is administered into the lumen of the lung of the subject in the amounts ranging from 0.001 to 100,000,000 Units; preferably 0.01 to 100,000 Units. One Unit of enzyme is defined as an amount of enzyme that will liberate 1.0 μmole of inorganic phosphate from adenosine 5′triphosphate per minute at 30° C. After initial dose, additional doses can be administered.

It is observed that the administration of soluble nucleotidase attenuates pulmonary edema and/or pulmonary inflammation as measured by improvement in at least one of the following parameters: depression of respiratory function, amount of epithelial lining fluid, alveolar fluid clearance, pulmonary edema as measured by radiographic visualization methods, level of oxygenation/hypoxia, FEV₁, FVC₁, length of a hospital stay, mortality or morbidity, increase in general quality of life, requirement for mechanical ventilation, the length of mechanical ventilation, the levels of inflammatory cells in the lung or outside of the lung in other anatomical compartments or spaces including systemic circulation, the amount of pro-inflammatory molecules including cytokines and chemokines in the lung or outside of the lung in other anatomical compartments or spaces including systemic circulation, pathological remodeling of the airway, patient-reported or physician-observed signs such as ease of breathing, or severity of coughing and/or wheezing, and feelings of well-being.

### Example 3

### Attenuation of Pulmonary Edema or Inflammation in Rat Model of Ventilator Induced Lung Injury

A rodent model of Ventilator Induced Lung Injury is set up as previously shown (Rich, et al., J. Trauma, 55:290-297 (2003); Douillet, et al. Am. J. Respir. Cell Mol. Biol. 32: 52-58 (2005)). Human recombinant nucleotidase NTPDase 3 was manipulated to express and purify as a soluble nucleotidase (Example 1). This soluble nucleotidase is administered by instillation into the lumen of the lung of the experimental animal in the amounts ranging from 0.01 Unit to 1000 Units. One Unit of enzyme is defined as an amount of enzyme that will liberate 1.0 μmole of inorganic phosphate from adenosine 5′triphosphate per minute at 30° C.

It is observed that the administration of soluble nucleotidase attenuates pulmonary edema and/or pulmonary inflammation as measured by improvement in at least one of the following parameters: depression of respiratory function, amount of epithelial lining fluid, wet to dry lung weight, alveolar fluid clearance, pulmonary edema as measured by radiographic visualization methods, level of oxygenation/hypoxia, mortality or morbidity, the levels of inflammatory cells in the lung or outside of the lung in other anatomical compartments or spaces including systemic circulation, the amount of pro-inflammatory molecules including cytokines and chemokines in the lung or outside of the lung in other anatomical compartments or spaces including systemic circulation, pathological remodeling of the airway, and observed signs such as ease of breathing.

### Example 4

### Attenuation of Pulmonary Edema or Inflammation in Rodent Model of Respiratory Syncytial Virus Infection

A rodent model of RSV infection is set up as previously shown (Davis, et al., Am. J. Physiol. Lung Cell Mol. Physiol. 286: L112-L120 (2004)). Human recombinant nucleotidase NTPDase 3 was manipulated to express and purify as a soluble nucleotidase (Example 1). This soluble nucleotidase is administered into the lumen of the lung of the experimental animal in the amounts ranging from 0.01 Unit to 1000 Units. One Unit of enzyme is defined as an amount of enzyme that will liberate 1.0 μmole of inorganic phosphate from adenosine 5′triphosphate per minute at 30° C.

It is observed that the administration of soluble nucleotidase attenuates pulmonary edema and/or pulmonary inflammation as measured by improvement in at least one of the following parameters: depression of respiratory function, amount of epithelial lining fluid, wet to dry lung weight, alveolar fluid clearance, pulmonary edema as measured by radiographic visualization methods, level of oxygenation/hypoxia, mortality or morbidity, the levels of inflammatory cells in the lung or outside of the lung in other anatomical compartments or spaces including systemic circulation, the amount of pro-inflammatory molecules including cytokines and chemokines in the lung or outside of the lung in other anatomical compartments or spaces including systemic circulation, pathological remodeling of the airway, and observed signs such as ease of breathing.

### Example 5

### Stability of Nucleotidases in Biological Fluid

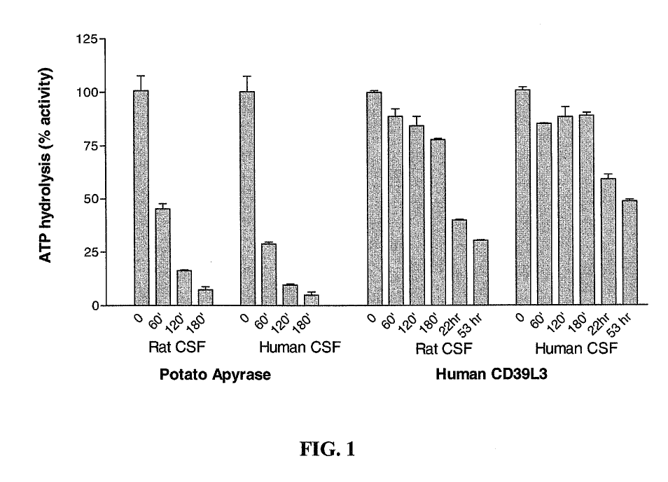

The stability of purified Grade VII potato apyrase (Sigma, St. Lois, Mo.) was compared to the stability of purified soluble NTPDase 3. This assay was performed in rat or human cerebro-spinal fluid (CSF) as the diluent. Stock solutions of each enzyme were prepared in 50 mM Tris buffer (pH 7.4) at a concentration of 1.0 unit per microliter. Reactions were incubated at 37° C., and were initiated with the addition of 10 microliters of enzyme stock solution into 90 microliters of CSF. At 0, 1, 2, 3, 22 and 53 hours, aliquots were removed for analysis. Enzymatic activity remaining in each reaction was determined by assessing the amount of Pᵢ-generated in 5 minutes at 37° C. from a 100 μM ATP solution. Percent activity for each reaction was compared to the activity present at time zero. The results are shown in FIG. 1.

Following incubation in human or rat CSF at 37° C., only small amount of potato apyrase was active in human and rat CSF after 3 hours (˜10%), while human recombinant soluble NTPDase 3 nucleotidase retained more then 50% of its activity after 53 hours as determined by ATP hydrolysis.

The invention, and the manner and process of making and using it, are now described in such full, clear, concise and exact terms as to enable any person skilled in the art to which it pertains, to make and use the same. It is to be understood that the foregoing describes preferred embodiments of the present invention and that modifications may be made therein without departing from the scope of the present invention as set forth in the claims. To particularly point out and distinctly claim the subject matter regarded as invention, the following claims conclude this specification.

1. A method for preventing or treating pulmonary edema and/or pulmonary inflammation in a human subject, comprising:
(a) identifying a human subject who is at risk of developing, or is suffering from pulmonary edema and/or pulmonary inflammation; and
(b) administering to said human subject at least one nucleotidase.

2. A method for treating pulmonary edema and/or pulmonary inflammation in a subject, comprising:
(a) identifying a subject who is suffering from pulmonary edema and/or pulmonary inflammation; and
(b) administering to said subject at least one nucleotidase; wherein said nucleotidase is not potato apyrase.

3. The method according to claim 2, wherein said nucleotidase is of a mammalian origin.

4. The method according to claim 2, wherein said nucleotidase is of a human origin.

5. The method according to claim 2, wherein said nucleotidase is soluble.

6. The method according to claim 5, wherein said nucleotidase is recombinant.

7. The method according to claim 2, wherein said nucleotidase is from NTPDase or NPP family.

8. The method according to claim 7, wherein said nucleotidase is from NTPDase family.

9. The method according to claim 8, wherein said nucleotidase is NTPDase 1, NTPDase 2, NTPDase 3, NTPDase 5, NTPDase 6, or NTPDase 8.

10. The method according to claim 7, wherein said nucleotidase is from NPP family.

11. The method according to claim 2, wherein said nucleotidase is glycosylated.

12. The method according to claim 1, wherein said method is for preventing or treating pulmonary edema.

13. The method according to claim 1, wherein said method is for preventing or treating pulmonary inflammation.

14. The method according to claim 2, wherein said pulmonary edema and/or pulmonary inflammation is caused by ventilator-induced lung injury or respiratory syncytial virus infection.

15. The method according to claim 2, wherein said method is for treating chronic obstructive pulmonary disease.

16. The method according to claim 2, wherein said administering is by local administration.

17. The method according to claim 16, wherein said local administration is inhalation.

18. The method according to claim 2, wherein said administering is by inhalation of aerosolized solution via nebulizer, by inhalation of dry powder via inhaler, or directing soluble or dried material into the air stream during mechanical ventilation.

19. A method of preventing or treating pulmonary edema and/or pulmonary inflammation comprising administering to a subject an effective amount of a nucleotidase selected from the group consisting of: adenosinetriphosphatase, apyrase, nucleoside-diphosphatase, ATP diphosphatase, nucleotide diphosphatase, ADP-ribose diphosphatase, nucleoside-triphosphatase, nucleoside-triphosphate diphosphatase, ADP-sugar diphosphatase, triphosphatase, thymidine-triphosphatase, UDP-sugar diphosphatase, phospholipid-translocating ATPase, magnesium-importing ATPase, cadmium-exporting ATPase, copper-exporting ATPase, zinc-exporting ATPase, proton-exporting ATPase, sodium-exporting ATPase, calcium-transporting ATPase, sodium/potassium-exchanging ATPase, hydrogen/potassium-exchanging ATPase, chloride-transporting ATPase, potassium-transporting ATPase, H(+)-transporting two-sector ATPase, sodium-transporting two-sector ATPase, arsenite-transporting ATPase, monosaccharide-transporting ATPase, oligosaccharide-transporting ATPase, maltose-transporting ATPase, glycerol-3-phosphate-transporting ATPase, polar-amino-acid-transporting ATPase, nonpolar-amino-acid-transporting ATPase, oligopeptide-transporting ATPase, nickel-transporting ATPase, sulfate-transporting ATPase, nitrate-transporting ATPase, phosphate-transporting ATPase, phosphonate-transporting ATPase, molybdate-transporting ATPase, Fe(3+)-transporting ATPase, polyamine-transporting ATPase, quatemary-amine-transporting ATPase, vitamin B12-transporting ATPase, iron-chelate-transporting ATPase, manganese-transporting ATPase, taurine-transporting ATPase, guanine-transporting ATPase, capsular-polysaccharide-transporting ATPase, lipopolysaccharide-transporting ATPase, teichoic-acid-transporting ATPase, heme-transporting ATPase, beta-glucan-transporting ATPase, peptide-transporting ATPase, xenobiotic-transporting ATPase, steroid-transporting ATPase, cadmium-transporting ATPase, fatty-acyl-CoA-transporting ATPase, alpha-factor-transporting ATPase, channel-conductance-controlling ATPase, protein-secreting ATPase, mitochondrial protein-transporting ATPase, chloroplast protein-transporting ATPase, Ag(+)-exporting ATPase, myosin ATPase, dynein ATPase, microtubule-severing ATPase, plus-end-directed kinesin ATPase, minus-end-directed kinesin ATPase, vesicle-fusing ATPase, peroxisome-assembly ATPase, proteasome ATPase, chaperonin ATPase, non-chaperonin molecular chaperone ATPase and nucleoplasmin ATPase.

20. The method according to claim 19, wherein the subject is a human subject.
